(19) United States
(12) Patent Application Publication
(10) Pub. No.: US 2025/0065034 A1
(43) Pub. Date: Feb. 27, 2025

(54) SECURING MEDICAL TUBING

(71) Applicant: Alera Medtech Inc., Oakville (CA)

(72) Inventors: Robert F. Rioux, Ashland, MA (US); Joshua A. Rose, Natick, MA (US)

(21) Appl. No.: 18/812,194

(22) Filed: Aug. 22, 2024

Related U.S. Application Data
(60) Provisional application No. 63/533,940, filed on Aug. 22, 2023.

(51) Int. Cl. A61M 5/14

(52) U.S. Cl. CPC A61M 5/14; A61M 2207/10; A61M 2209/088

(57) ABSTRACT

A catheter stabilization device is configured and used to secure medical tubing to a location on a body of a patient. When the device is employed, the tubing is maintained in such a manner that unintentional disruption to or removal of an infusion port from an infusion site is eliminated or at least minimized.

### CROSS-REFERENCE TO RELATED APPLICATIONS

This application claims priority to, and the benefit of, U.S. Provisional Application No. 63/533,940, filed on Aug. 22, 2023, the content of which is incorporated by reference herein in its entirety.

### TECHNICAL FIELD

The invention generally relates to devices for securing or stabilizing one or more medical tubes.

### BACKGROUND INFORMATION

Tubing of a variety of different sizes are used in various medical procedures to treat humans and/or other types of mammals. For example, intravenous infusion uses one or more catheters. Devices for securing or stabilizing medical tubes to one or more locations on a patient's body are known. For example, Becton, Dickinson and Company (“BD” which is headquartered in Franklin Lakes, New Jersey in the United States) has a line of StatLock™ stabilization devices that BD claims on a page of its website are a more effective alternative to tape in helping improve clinical outcomes, quality of care and economic efficiencies. BD also claims on its website that its StatLock™ devices, which are available in adult and pediatric sizes, offer needle-free securement of chest tubes, sheath introducers, drainage devices and more, through the use of nylon strands that grip the tubing. As another example, Alera Medtech Inc. (“Alera” of Oakville, Ontario in Canada) has its SureSet™ Safe-Track Strain Relief Technology™ that Alera claims on a page of its website creates a streamlined system to reliably stabilize a peripheral intravenous line (PIV) catheter and organize the infusion tubing. Some published patent documents that relate to catheter stabilization devices are owned by BD or Alera including, for example, International Patent Application Publication No. WO 2022/056376 A1, issued U.S. Pat. No. 9,878,129, and issued U.S. Pat. Nos. 6,582,403, 6,770,055, 7,837,655, 7,223,256, 7,635,355, 7,785,295, 7,014,627, 7,799,001, 7,762,991, 8,357,124, 8,394,065, 8,636,698, 9,561,348, D629,514, D613,857, and D613,858. Another known published patent document is U.S. Patent Application Publication No. US 2019/0351185 A1.

### SUMMARY OF THE INVENTION

Taking intravenous infusion as one exemplary medical procedure that uses one or more tubes in the treatment of a patient (whether the patient is a human or some other type of mammal), it is noted that intravenous infusion is a therapeutic treatment of the patient that involves the introduction of a liquid, such as a saline solution, into a vein of the patient. The intravenous infusion is introduced into a particular location of a body of the patient (referred to as the infusion site or the insertion site) through an infusion port that generally has a needle suitable for accessing the patient's vein. Tubing extends from the infusion port outside of the patient's body to a fluid receptacle (such as a sealed plastic bag) that also is located outside of the patient's body. Intravenous infusion can be done in an outpatient environment or office, as an inpatient procedure, in a surgical operating room, or in a long-term care facility, and it also can be done while the patient is sedated or not sedated. A sedated patient may make involuntary movements that have the potential of disrupting the infusion port from the infusion site or else possibly completely removing the infusion port from the infusion site. Voluntary movement by a non-sedated patient also may displace the infusion port from the infusion site. When a device according to the invention is used, displacement of the tubing and/or the infusion port from the infusion site becomes less likely. The inventive device allows typical movements by the patient (whether sedated or not) without the tubing or the infusion port becoming disrupted or removed from the infusion site. A device according to the invention can provide the same medical tubing placement security when used in connection with other medical procedures including, for example, urine drainage from a patient by use of a Foley catheter.

In one aspect, the invention involves a catheter stabilization device that has a movable member that allows the device to accommodate a variety of catheters (or other types of medical tubing) of different sizes. For example, the very same inventive device could hold and secure a tube with an outer diameter of 9 Fr (where Fr, or sometimes simply F, represents a French unit, which is the same as an outer diameter of 3 millimeters), a tube with an outer diameter of 15 Fr (which is an outer diameter of 5 millimeters), or a tube of any outer diameter between 9 Fr and 15 Fr. As another example, a device according to the invention instead could hold and secure a tube with an outer diameter of 5 millimeters (which is 15 Fr) and up to and including 7 millimeters (which is 21 Fr). Those two ranges (of 3 mm to 5 mm, which could be used for a female human patient, and of 5 mm to 7 mm, which could be used for a male human patient) are exemplary of tubing outer diameter ranges that a device according to the invention could handle (that is, hold and secure in place) because of the movable member that is part of the inventive device. As another example, a device according to the invention could hold and secure a tube or catheter with an outer diameter of 2 millimeters and up to and including 4 millimeters, or another tubing outer diameter range such as, for example, 1.33 millimeters to 3 millimeters, or as another example 4 millimeters to 6 millimeters, or as yet another example 5 millimeters to 8 millimeters. Other outer diameter ranges also are possible, and the outer diameter range difference could be, for example, 2 millimeters (to accommodate, for example, the tubing of a Foley catheter that can range from 6 French, which is 2 mm, to 12 French, which is 4 mm), less than 2 millimeters (such as about 0.7 mm, to accommodate, for example, the tubing of a Foley catheter that can range from 16 French, which is about 5.33 mm, to 18 French, which is 6 mm), or more than 2 millimeters (such as about 2.7 mm, to accommodate, for example, the tubing of a Foley catheter that can range from 16 French, which is about 5.33 mm, to 24 French, which is 8 mm). Again, it primarily is the unique movable member of the device that allows the inventive device to hold and secure any of a variety of different outer diameter catheters within a certain tubing outer diameter range.

The movable member can be formed integrally with and as part of the inventive device, and the entire device can be made as a single piece and of some type of plastic, by for example an injection molding process which is well known in the field of manufacturing certain medical devices. The single-piece device can be mated with a base or support structure (by, for example, ultrasonic welding, which also is well known in the field of manufacturing certain medical devices), and the support structure can include an adhesive layer or coating that can be attached directly to the skin of a patient to hold the entire structure in place at a desired location of the patient's body such as somewhere on an arm, the chest, or a thigh of the patient.

A device according to the invention can be, as indicated immediately above, created as a single-piece device (by, for example, using plastics injection molding technology) and then that single-piece device can be attached to a base or support structure (by, for example, using ultrasonic welding technology) to complete the entire medical device that then can be removably attached to the skin of the patient for use as a medical tubing securement device. Such an inventive device can have a section configured to receive at least a portion of the catheter or other medical tubing that is going to be used as part of the patient's treatment. The device also can have a member configured to move when at least a portion of the portion of the tubing that is received by the section is pressed against the member. And, finally, the device can have a cover that is configured to apply pressure to keep the portion of the tubing within the section when the cover is closed. The movable member gets deflected from one position to another position as a result of the pressure applied to the received portion of the tubing when the cover of the device is closed. The device can use a male clip and a female tab configuration to mechanically connect and thereby hold the cover closed even though the retained tubing, if large enough in outer diameter, is pushing back and trying to pop open the cover.

These and other aspects and details of devices according to the invention will become clearer by referring to the following parts of this document, and various methods of making and using such devices will become clearer as well. The entirety of this document illustrates devices and methods that embody the invention, but the invention is not limited only to the specific devices and methods disclosed herein. Various ideas and combinations that can be derived from the contents of this document (including both the text and the accompanying drawings) are to be considered as disclosed and included herein.

### BRIEF DESCRIPTION OF THE DRAWINGS

FIGS. 1-8 show a first embodiment of a catheter stabilization device in accordance with the invention. FIG. 1 is a top perspective view with the device in an open position. FIG. 2 is a bottom perspective view with the device also in the open position. FIG. 3 shows an infusion port that has been placed by a medical professional (such as a nurse) at an infusion site of a patient, and FIG. 3 also shows the catheter stabilization device before it has been placed on the patient at or near the infusion site. FIG. 4 shows the device placed on the patient over the infusion site. FIG. 5 shows tubing that runs from the infusion port outside of the patient's body, with a portion of the tubing placed within a trench of the device. FIG. 6 shows the device with its cover closed to retain that portion of the tubing within the trench. FIG. 7 shows the closed catheter stabilization device in cross section, with the device having a first type of movable member. FIG. 8 shows the same closed device in cross section, but with the device having a second type of movable member.

FIGS. 9-14 show a second embodiment of a catheter stabilization device in accordance with the invention. FIG. 9 is a top perspective view with the device in an open position. FIG. 10 is a bottom perspective view with the device also in the open position. FIG. 11 shows an infusion port that has been placed by a medical professional (such as a nurse) at an infusion site of a patient, and FIG. 11 also shows the catheter stabilization device before it has been placed on the patient at or near the infusion site. FIG. 12 shows the device placed on the patient near the infusion site with a portion of tubing (that runs from the infusion port outside of the patient's body) within a trench of the device. FIG. 13 shows the device with its cover closed to retain that portion of the tubing within the trench. FIG. 14 shows the closed catheter stabilization device in cross section, with the device having the first type of movable member.

Each of these drawings is referenced again and explained in more detail below.

### DETAILED DESCRIPTION

It is noted initially that Alera Medtech Inc. (of Oakville, Ontario in Canada) is the owner of U.S. Pat. No. 9,878,129 and International Patent Application Publication No. WO 2022/056376 A1. Those two published patent documents are directed to maintaining tubing with certain retainer devices and by certain methods, and the entirety of each of the two published patent documents (including text and drawings) is hereby incorporated by reference into this document.

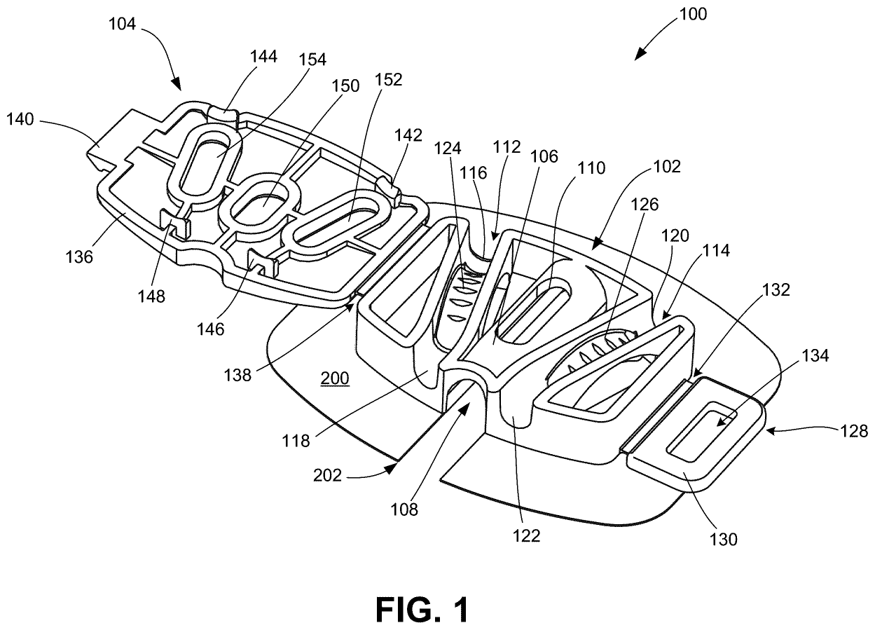

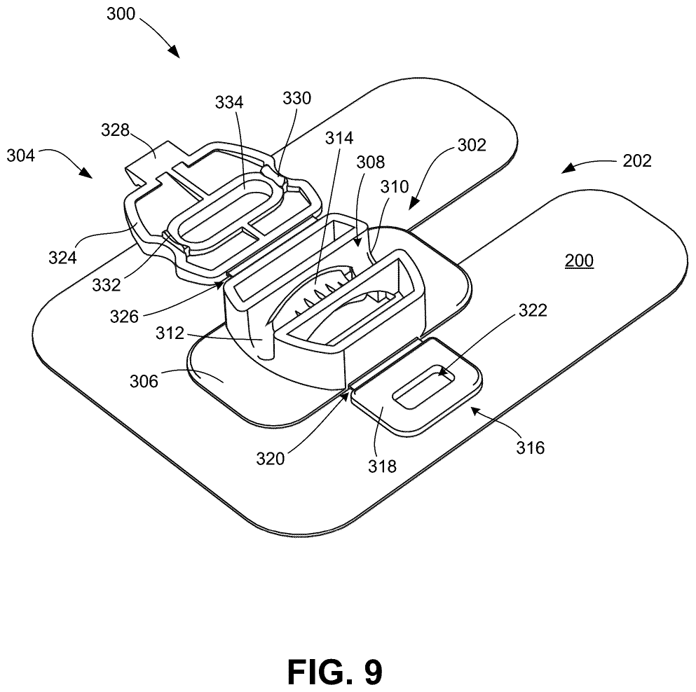

Turning now to the drawings that are directed to the present invention, FIGS. 1-8 that accompany this text show a first embodiment of a device 100 and method in accordance with the invention, and FIGS. 9-14 show another embodiment of a device 300 and method in accordance with the invention. The invention is not limited only to these two specific embodiments, and various combinations of the features and aspects of the described and illustrated devices and methods are to be considered disclosed herein even if not specifically called out.

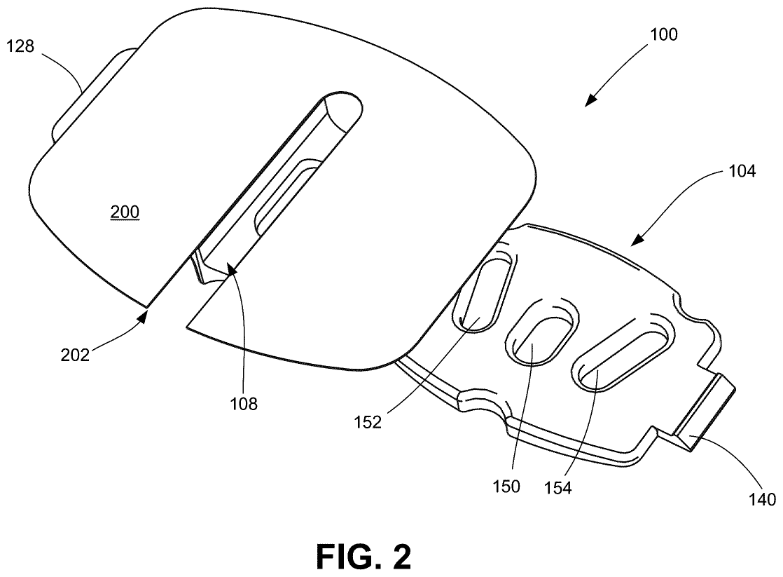

FIG. 1 is a top perspective view of one embodiment of the device 100, with the device 100 in an open position, and FIG. 2 is a bottom perspective view of the device 100 of FIG. 1, also with the device 100 in the open position. The device 100 also can be referred to as, for example, a stabilization device, a catheter stabilization device, a medical tubing securement device, a tubing stabilization device, or a retainer. The device 100 can have a base 102 and a cover 104.

To make it practical for use with a patient, the device 100 typically will have a support structure 200. The support structure 200 can be attached on one of its extended surfaces to the bottom of the base 102 (by, for example, ultrasonic welding), and the opposite surface of the support structure 200 can be configured for removable attachment to the skin of a patient, by for example an adhesive that is on that opposite side of the support structure 200. The support structure 200 typically will have a cutout or opening 202, as seen in FIGS. 1-3. The opening 202 in the support structure 200 is for accommodating medical tubing that is external to the patient (such as the tubular part of a Foley catheter that is external to the patient and used to carry urine from the patient's bladder to outside of the patient's body) and/or for accommodating an infusion luer and/or an intravenous hub connection, for example.

The support structure 200 can be formed of multiple layers, and in one example the support structure 200 can have a middle clear plastic layer sandwiched between an upper fabric layer and a lower cover layer. The lower cover layer can be a clear plastic film. A bottom surface of the upper fabric layer can be configured to be attached to a top surface of the middle clear plastic layer, and a bottom surface of the middle clear plastic layer can be configured to be attached to a top surface of the lower cover layer. The bottom surface of the middle clear plastic layer can have an adhesive on it which, prior to use of the device 100/support structure 200 combination, will be covered by the lower cover layer. A medical professional (such as a nurse) or other person can easily remove the lower cover layer by peeling it off of the bottom of the middle layer to expose the adhesive on the bottom surface of the middle clear plastic layer and thus allow that adhesive surface to be attached to the skin of a patient at some location on the patient's body such as the arm or thigh of the patient, for example.

Still referring to FIG. 1, the base 102 of the device 100 can be seen generally to have a top surface and an opposite bottom surface. The bottom surface of the base 102 can have an adhesive on it for attachment to the top surface of the support structure 200, and/or the bottom surface of the base 102 and the top surface of the support structure 200 can be attached to each other by ultrasonic welding or some other attachment technique. Regardless of how they are attached, the goal is to connect the bottom surface of the base 102 to the top surface of the support structure 200 in a manner that makes it at least difficult and/or unlikely for those two surfaces to become unattached from each other during typical handling and use of the device 100/supporting structure 200 combination (both before placement on a patient and also after placement on a patient).

In the embodiment of the device 100 shown in FIGS. 1-8, there are two passageways or trenches formed in the base 102, namely a first trench 112 and a second trench 114. The first trench 112 has a first end 116 and an opposing second end 118, and the second trench has a first end 120 and an opposing second end 122. Importantly, a movable member is contained within each of the first and second trenches 112, 114. A first movable member 124 is disposed in the first trench 112, and a second movable member 126 is disposed in the second trench 114. The movable members 124, 126 in the trenches 112, 114 are an important part of what makes the device 100 able to accommodate (that is, hold and secure in place) any one of a variety of different-diameter catheters (or other types of medical tubing). For example, one exemplary device according to the invention could hold and secure an infusion tube in either trench (or both trenches) with an outer diameter of 2 millimeters, and that very same device also could hold and secure in either trench (or both trenches) an infusion tube with an outer diameter of 4 millimeters as well as any other infusion tube with an outer diameter between 2.0 mm and 4.0 mm. Another exemplary inventive device could hold/secure in either trench (or both trenches) infusion tubing of a different outer diameter range such as, for example, 4 millimeters to 6 millimeters or else, as another example, 1 millimeter to 3 millimeters. It also is possible for each of the trenches of the device to accommodate (that is, hold and secure in place) tubing of a certain outer diameter range that is different. For example, a first trench of the device could accommodate any tube with an outer diameter from 2.0 mm to 4.0 mm, while a second trench of the device could accommodate any tube with an outer diameter from 1.0 mm to 3.0 mm. It is noted that while the device 100 is shown having two trenches, it is possible to have an inventive device with more than two trenches and thus more than two movable members.

As seen in FIGS. 1 and 2, a body 136 of the cover 104 of the device 100 has three openings or viewing windows 150, 152, 154. The viewing window 150 in roughly the middle or center of the cover 104 is configured to align (when the cover 104 is closed) with a middle or center viewing window 110 formed in the base 102 of the device 100, and other two viewing windows 152, 154 formed in the cover 104 are configured to align (again, when the cover 104 is closed) with, respectively, the first and second trenches 112, 114 of the base 102. Also, part of the cover 104 is a clip 140 which is integral with and extends from the body 136 of the cover 104. The clip 140 is configured to enter an opening or aperture 134 formed in the body 130 of a hinged securement tab 128 of the device 100.

Each of the hinged securement tab 128 and the cover 104 is movable with respect to the base 102 because of a hinge, namely living hinge 132 and living hinge 138, respectively. Integral hinge is another known term that is used for living hinge. A living hinge (or an integral hinge) is a thin flexible hinge that is flexure bearing and that typically is made from the same material as the two pieces that the hinge connects, and it is known to form such hinges by using an injection molding process to create a single-piece unit made of the same material(s). It is possible to use other types of hinges or other mechanical arrangements to make the tab 128 and the cover 104 movable with respect to the base 102, but using a living hinge is one way that also has the benefit of allowing the device 100 to be made as a single-piece unit.

The device 100 of FIGS. 1-8 can be made as a single-piece unit from a plastic material by using an injection molding process. A medical grade plastic typically would be used in such a process of making the device 100. As one example, polyethylene is a common medical grade plastic polymer that can be used in an injection molding process to create a medical device. The support structure 200 of FIGS. 1-8 can be made by a manufacturing process that is separate from the process used to make the device 100. The device 100 and the support structure 200 then can be attached to each other in any of a variety of ways to form the combined device 100/support structure 200 unit. One way of attaching the device 100 to the support structure 200 is to use an adhesive to attach at least a portion of the bottom surface of the base 102 of the device 100 to at least a portion of the top surface of the support structure 200. Another way would be to use ultrasonic welding to attach at least a portion of the bottom surface of the base 102 of the device 100 to at least a portion of the top surface of the support structure 200. One or more other ways known in the medical device manufacturing business could be used to attach the device 100 to the support structure 200.

The cover 104 also has two channel brackets 142, 146 that will be aligned with the first and second ends 116, 118 of the first trench 112 when the cover 104 is closed. And the two other channel brackets 144, 148 will be aligned with the first and second ends 120, 122 of the second trench 114 when the cover 104 is closed. The purpose of these channel brackets is described later, below.

Still referring to FIGS. 1 and 2, a semi-cylindrical protrusion 106 in the base 102 of the device 100 forms a tunnel 108 in the base 102. The tunnel 108 is for accommodating medical tubing that is external to the patient (such as the tubular part of a Foley catheter that is external to the patient and used to carry urine from the patient's bladder to outside of the patient's body) and/or for accommodating an infusion luer and/or an intravenous hub connection, for example.

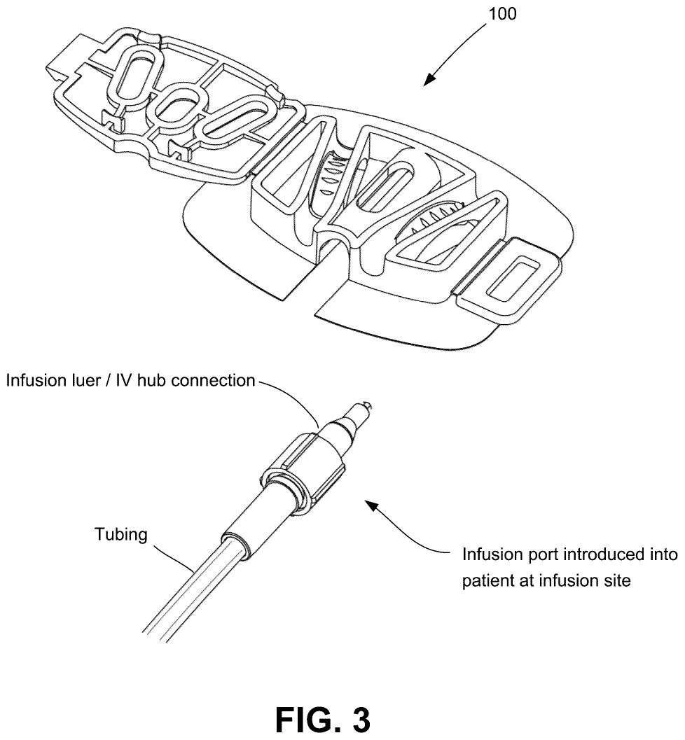

FIG. 3 shows an infusion port that has been placed by a medical professional (such as a nurse) at an infusion site (also known as an insertion site) of a patient, and FIG. 3 also shows the device 100 of FIG. 1 before it has been placed on the patient at or near that infusion site. The needle of the infusion port that is shown in FIG. 3 (which would have been at the end of the infusion port opposite from the tubing) has already been inserted into the body of a patient, and that is why the needle itself is not visible in FIG. 3. With the infusion port in place at the desired location of the patient's body, such as in the patient's arm or in the top part of the patient's hand, the combination of the device 100 and its support structure 200 can be placed on the patient's skin at and over the infusion site.

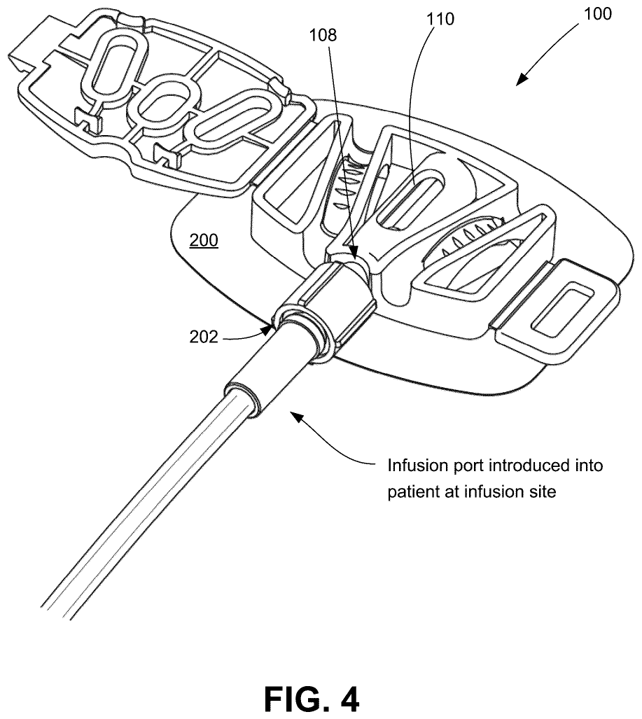

FIG. 4 shows the device 100 of FIG. 1 placed on the patient's skin over the infusion site. With the support structure 200 of the device 100 stuck to the patient's skin at and over the infusion site, the infusion site is exposed to the eyes of a medical professional or other person via the viewing window 110 formed in the base 102 of the device 100. The infusion luer/intravenous hub connection of the infusion port is received and accommodated within the tunnel 108 of the base 102 of the device 100, as shown in FIG. 4. And, similarly, the opening 202 in the support structure 200 receives and accommodates the infusion port, as also shown in FIG. 4.

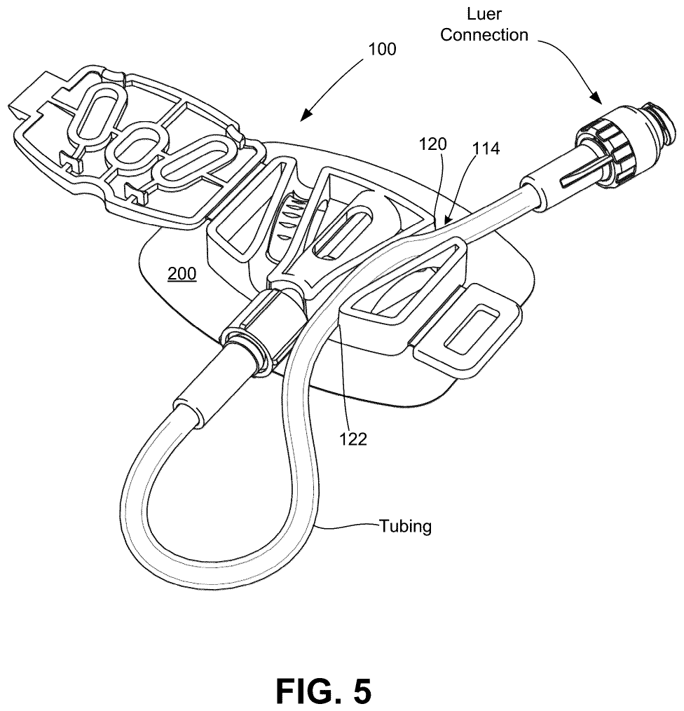

FIG. 5 is similar to FIG. 4 but FIG. 5 shows tubing that runs from the infusion port outside of the patient's body, with a portion of the tubing placed within the second trench 114 of the device 100. The tubing easily could have been placed within the first trench 112 of the device 100 instead, but it is shown in FIG. 5 as having been placed in the second trench 114. It is noted that the medical professional or other person that places the tubing within the trench (whether it is the first trench 112 and/or the second trench 114) does not need to push down into or onto the patient with much, or possibly any, force at all to place the tubing within the trench(es), and this is because of, and a benefit that results from, the device 100 having the movable members 124, 126, with the end result being much more comfort for the patient as opposed to known tubing retention devices that are available commercially and that typically require a greater amount of force to be used to get the tubing into a trench of the device.

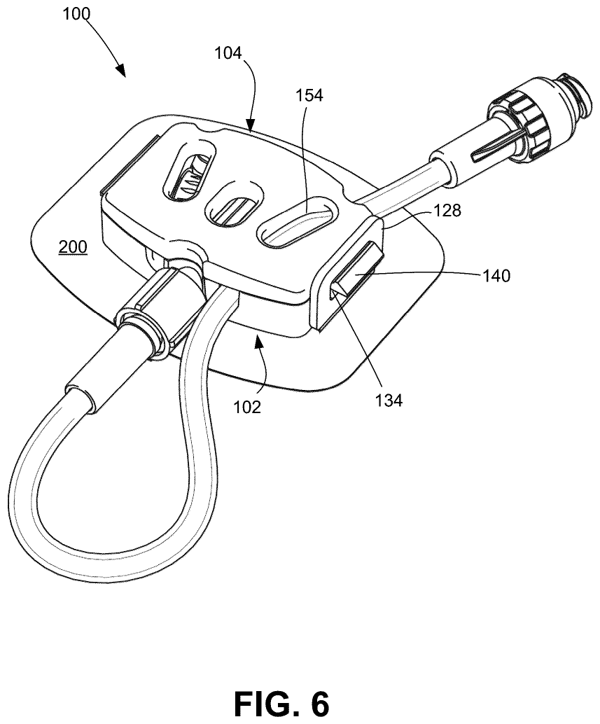

FIG. 6 is similar to FIG. 5 but FIG. 6 shows the device 100 with its cover 104 closed to retain the portion of the tubing that has been placed within the trench 114 of the device 100. In FIGS. 5 and 6, a luer connection on the end of the tube opposite from the infusion port is shown for the first time, and it is noted that this luer connection can be attached to a compatible connection to run more tubing or something else from that luer connection to deliver and/or remove some type of fluid to and/or from the patient. With the cover 104 of the device 100 closed, as shown in FIG. 6, the viewing window 154 shows the tubing retained within the second trench 114, and the viewing window 152 shows that there is nothing in the first trench 112 of the device 100.

Once the cover 104 is placed over the base 102, as shown in FIG. 6, the person closing the device 100 will hold the cover 104 down on the base 102 with one hand and, either with that same hand or with their other hand, will move the hinged securement tab 128 so that the clip 140 of the cover 104 passes through the aperture 134 formed in the body 130 of the hinged securement tab 128. With the cover 104 closed in this way, the cover 104 is held securely in its closed position, and it is unlikely that any force pushing up on the cover 104 by the tubing retained in the second trench 114 will result in the cover 104 unexpectedly or unintentionally popping open. Also, the hinged side movement of the tab 128 to get the clip 140 to enter and be retained within the aperture 134 in the tab 128 means that force to close the cover 104 is a side force by the person placing the device 100, and this closing side force is easier for the placing person to apply as well as more comfortable for the patient on which the device 100 has been placed. With known retainer devices, a downward force (into the patent and the patient's skin) is required which is harder for the placing person to do and is less comfortable to the patient.

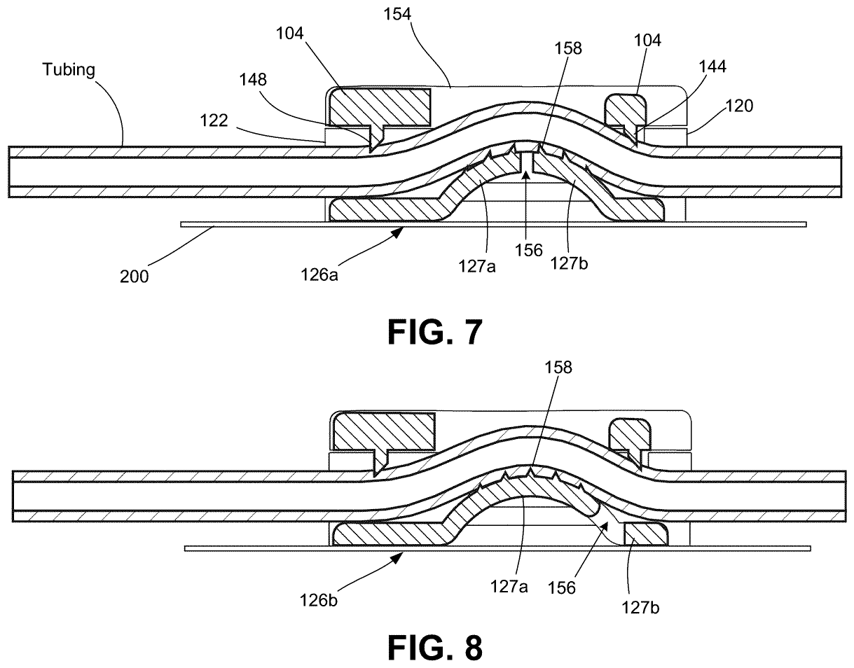

FIG. 7 shows the device 100 of FIG. 6 in cross section, but with the device 100 having a movable member 126a of a type different from the type of movable member 126, 126b shown in FIGS. 1-6 and 8. The movable member 126a can be seen and understood to be a two-piece or two-portion 127a, 127b bridge with a slit or separation 156 between the two pieces 127a, 127b. The slit 156 and the fact that the two pieces 127a, 127b are not connected means that a force pushing down on the two pieces 127a, 127b will cause the two pieces 127a, 127b to move down (toward the support structure 200) further into the second trench 114, at least until the slit 156 is closed by the downward movement of the two pieces 127a, 127b. This movability of the movable member 126a is important in the device 100 being able to accommodate any one of a variety of different-diameter tubes (whether catheters or other types of medical tubing). A device 100 with the movable member 126a can accommodate and hold secure tubing with a certain maximum outer diameter, with the cover 104 closed and not popping open, and that same device 100 with that movable member 126a also can accommodate and hold secure tubing with an outer diameter less than that maximum diameter. There is a range of outer diameters of tubing that such a device 100 can accommodate and hold secure, and this then means that a different specific device 100 does not have to be made for each specific tubing outer diameter. For example, one exemplary device according to the invention could hold and secure an infusion tube with an outer diameter of 2 millimeters, and that very same device also could hold and secure an infusion tube with an outer diameter of 4 millimeters as well as any other infusion tube or other type of tubing or catheter with an outer diameter between 2.0 mm and 4.0 mm (which is 6 Fr to 12 Fr). Other tubing outer diameter ranges can be accommodated by an exemplary inventive device such as, for example, 4 millimeters to about 5.33 millimeters (which is 12 Fr to 16 Fr), 4 millimeters to 6 millimeters (which is 12 Fr to 18 Fr), 5 millimeters to 7 millimeters (which is 15 Fr to 21 Fr), 5 millimeters to 8 millimeters (which is 15 Fr to 24 Fr), about 5.33 millimeters to 6 millimeters (which is 16 Fr to 18 Fr), or about 6.67 millimeters to 8 millimeters (which is 20 Fr to 24 Fr).

As mentioned above, the cover 104 has two channel brackets 142, 146 that will be aligned with the first and second ends 116, 118 of the first trench 112 when the cover 104 is closed. The cover 104 also has two other channel brackets 144, 148 that will be aligned with the first and second ends 120, 122 of the second trench 114 when the cover 104 is closed. The purpose of these channel brackets can be seen with reference to FIG. 7. Channel brackets 144, 148 contact the tubing and push down on or into the wall of the tubing to hold it in place in the second trench 114 when the cover 104 of the device 100 is closed. That force pushing down on the tubing by the closed cover 104 and the channel brackets 144, 148 also causes the tubing to push down on the two pieces 127a, 127b of the movable member 126a. In addition to the channel brackets 144, 148 contacting and pushing down on or into the wall of the tubing when the cover 104 is closed, the channel brackets 144, 148 help to prevent longitudinal movement of the tubing because of how they contact and/or dig into a portion of the wall of the tubing, as shown in FIG. 7. It is noted that the other two channel brackets 142, 146 will perform the same function on any tubing that might be within the first trench 112 when the cover 104 is closed.

With reference still to FIG. 7, it is noted that one or both of the two pieces 127a, 127b can have one or more protrusions, barbs, or gripping members 158 on its surface that face/contact the tubing. Such gripping members 158 can perform a similar function as the channel brackets 144, 148 (or 142, 146), in that the gripping member(s) 158 can contact and push up on or into a portion of the wall of the tubing to hold the tubing in place within the trench and to prevent longitudinal movement of the tubing within the trench. Each gripping member 158 can be oriented in a direction that is left, up, or right, depending on the type or direction of gripping that is desired for each of the gripping members 158. The gripping members 158 shown in FIG. 7 are pointed generally in the up direction.

FIG. 8 shows the device 100 of FIG. 6 in cross section, with the same type of movable member 126, 126b as seen in FIGS. 1-6. Unlike the movable member 126a of FIG. 7, the movable member 126b of FIG. 8 can be seen and understood to be a bridge with one long piece 127a separated by a slit 156 from the other end of the bridge 127b. This can be viewed as a bridge with one connection point on one end of the bridge (as shown in FIG. 8) versus a bridge with two connection points where one is at one end of the arched bridge and the other is at the other/opposite end of the arched bridge (as shown in FIG. 7). A one-piece or one-portion 127a bridge with a slit or separation 156 at one end of the bridge is shown in FIG. 8.

In FIG. 8, a force pushing down on the one long piece 127a of the bridge will cause that long piece 127a to move down (toward the support structure 200) further into the second trench 114, at least until the space of the slit 156 is closed by the downward movement of that one long piece 127a. This movability of the movable member 126b (like the movability of the two-piece bridge 126a in FIG. 7) is important in the device 100 being able to accommodate any one of a variety of different-diameter tubes (whether catheters or other types of medical tubing). A device 100 with the movable member 126b can accommodate and hold secure tubing with a certain maximum outer diameter, with the cover 104 closed and not popping open, and that same device 100 with that movable member 126b also can accommodate and hold secure tubing with an outer diameter less than that maximum diameter. There is a range of outer diameters of tubing that such a device 100 can accommodate and hold secure, and this then means that a different specific device 100 does not have to be made for each specific tubing outer diameter. For example, one exemplary device according to the invention could hold and secure an infusion tube with an outer diameter of 2 millimeters, and that very same device also could hold and secure an infusion tube with an outer diameter of 4 millimeters as well as any other tube with an outer diameter between 2.0 mm and 4.0 mm. Other outer tubing diameter ranges can be accommodated by an exemplary inventive device such as, for example, 4 millimeters to 6 millimeters or any of a variety of other ranges such as the ones given above (whether expressed in millimeters and/or French units).

The one long piece 127a of FIG. 8 can have one or more gripping members 158 on it, and each gripping member 158 can be oriented in a direction that is left, up, or right, depending on the type or direction of gripping that is desired for each of the gripping members 158. The gripping members 158 shown in FIG. 8 are pointed generally in the up direction.

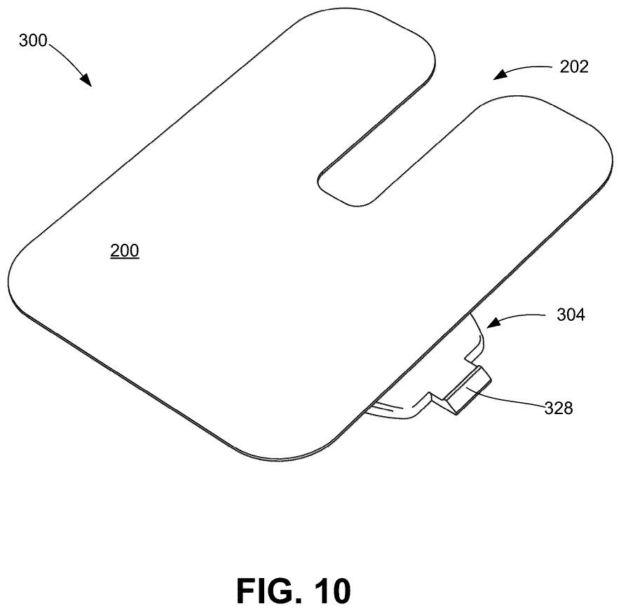

FIG. 9 is a top perspective view of another embodiment of a device according to the invention. The device 300 is shown in FIG. 9 in an open position. FIG. 10 is a bottom perspective view with the device 300 of FIG. 9, also in the open position. Like the device 100 of FIGS. 1-8, the device 300 of FIGS. 9-14 also can be referred to as a stabilization device, a catheter stabilization device, a medical tubing securement device, a tubing stabilization device, or a retainer, for example. The device 300 can have a base 302 and a cover 304, as shown in FIGS. 9-14.

As with the device 100 of FIGS. 1-8, the device 300 of FIGS. 9-14 typically will have the support structure 200 to make it practical for use with a patient. The support structure 200 can be attached on one of its extended surfaces to the bottom of the base 302 (by, for example, ultrasonic welding), and the opposite surface of the support structure 200 can be configured for removable attachment to the skin of a patient, by for example an adhesive that is on that opposite side of the support structure 200. The support structure 200 seen in FIGS. 9-13 typically will have the same cutout or opening 202 as seen in FIGS. 1-3. The opening 202 in the support structure 200 of FIGS. 9-13 is for accommodating medical tubing that is external to the patient (such as the tubular part of a Foley catheter that is external to the patient and used to carry urine from the patient's bladder to outside of the patient's body) and/or for accommodating an infusion luer and/or an intravenous hub connection, for example. The support structure 200 can be formed of multiple layers, just as described previously.

With reference to FIG. 9, the base 302 of the device 300 can be seen generally to have a top surface and an opposite bottom surface. The bottom surface of the base 302 can have an adhesive on it for attachment to the top surface of the support structure 200, and/or the bottom surface of the base 302 and the top surface of the support structure 200 can be attached to each other by ultrasonic welding or some other attachment technique. Regardless of how they are attached, the goal is to connect the bottom surface of the base 302 to the top surface of the support structure 200 in a manner that makes it at least difficult and/or unlikely for those two surfaces to become unattached from each other during typical handling and use of the device 300/supporting structure 200 combination (both before placement on a patient and also after placement on a patient).

In the embodiment of the device 300 shown in FIGS. 9-14, there is one passageway or trench formed in the base 302, namely a first trench 308. The trench 308 has a first end 310 and an opposing second end 312. Importantly, a movable member 314 is contained within the trench 308. The movable member 314 that is disposed in the trench 308 is an important part of what makes the device 300 able to accommodate any one of a variety of different-diameter catheters (or other types of medical tubing). For example, one exemplary device according to the invention could hold and secure the tube of a Foley catheter with an outer diameter of 5 millimeters, and that very same device also could hold and secure the tube of a Foley catheter with an outer diameter of 6 millimeters as well as any other tube of a Foley catheter with an outer diameter between 5.0 mm and 6.0 mm. Other outer tubing diameter ranges can be accommodated by an exemplary inventive device such as, for example, 3 millimeters to 5 millimeters, or, as another example, 5 millimeters to 7 millimeters, or, as still further examples, any of the outer tubing ranges provided above (whether expressed in millimeters and/or French units).

The base 302 of the device 300 has a flange 306 as part of the bottom portion of the device, and this flange 306 is there primarily to provide additional surface area to the bottom of the base of the device 300 given that the device 300 of FIGS. 9-14 is about half the footprint of the device 100 of FIGS. 1-8 without the flange 306. The flange 306 gives the device 300 more area to contact the support structure 200 than device 300 otherwise would have without the flange 306.

As seen in FIGS. 9 and 10, a body 324 of the cover 304 of the device 300 has one opening or viewing window 334. This viewing window 334 of the device 300 is configured to align (when the cover 304 is closed) with the trench 308 of the base 302 of the device 300. Also, part of the cover 304 is a clip 328 which is integral with and extends from the body 324 of the cover 304. The clip 328 is configured to enter an opening or aperture 322 formed in the body 318 of a hinged securement tab 316 of the device 300.

Each of the hinged securement tab 316 and the cover 304 is movable with respect to the base 302 because of a hinge, namely living hinge 320 and living hinge 326, respectively. Integral hinge is another known term that is used for living hinge. A living hinge (or an integral hinge) is a thin flexible hinge that is flexure bearing and that typically is made from the same material as the two pieces that the hinge connects, and it is known to form such hinges by using an injection molding process to create a single-piece unit made of the same material(s). It is possible to use other types of hinges or other mechanical arrangements to make the tab 316 and the cover 304 movable with respect to the base 302, but using a living hinge is one way that also has the benefit of allowing the device 300 to be made as a single-piece unit.

The device 300 of FIGS. 9-14 can be made as a single-piece unit from a plastic material by using an injection molding process. A medical grade plastic typically would be used in such a process of making the device 100. As one example, polyethylene is a common medical grade plastic polymer that can be used in an injection molding process to create a medical device. The support structure 200 of FIGS. 9-14 can be made by a manufacturing process that is separate from the process used to make the device 300. The device 300 and the support structure 200 then can be attached to each other in any of a variety of ways to form the combined device 300/support structure 200 unit. One way of attaching the device 300 to the support structure 200 is to use an adhesive to attach at least a portion of the bottom surface of the base 302 of the device 300 to at least a portion of the top surface of the support structure 200. Another way would be to use ultrasonic welding to attach at least a portion of the bottom surface of the base 302 of the device 300 to at least a portion of the top surface of the support structure 200. One or more other ways known in the medical device manufacturing business could be used to attach to the device 300 to the support structure 200.

The cover 304 also has two channel brackets 330, 332 that will be aligned with the first and second ends 310, 312 of the trench 308 when the cover 304 is closed. The purpose of these channel brackets is described later, below.

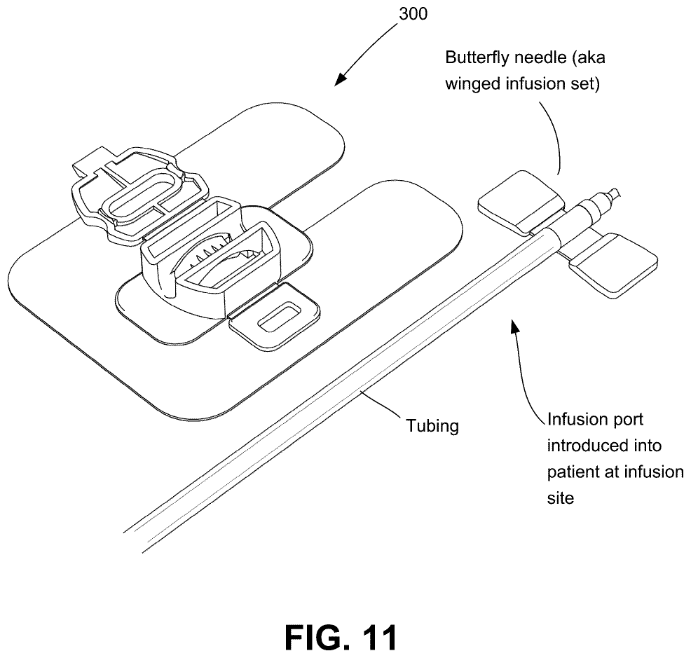

FIG. 11 shows an infusion port that has been placed by a medical professional (such as a nurse) at an infusion site (also referred to as an insertion site) of a patient, and FIG. 11 also shows the device 300 of FIG. 9 before it has been placed on the patient at or near the infusion site. The needle of the infusion port that is shown in FIG. 11 (which would have been at the end of the infusion port opposite from the tubing) has already been inserted into the body of a patient, and that is why the needle itself is not visible in FIG. 11. With the infusion port in place at the desired location of the patient's body, such as in the patient's arm or in the top part of the patient's hand, the combination of the device 300 and its support structure 200 can be placed on the patient's skin at or near the infusion site.

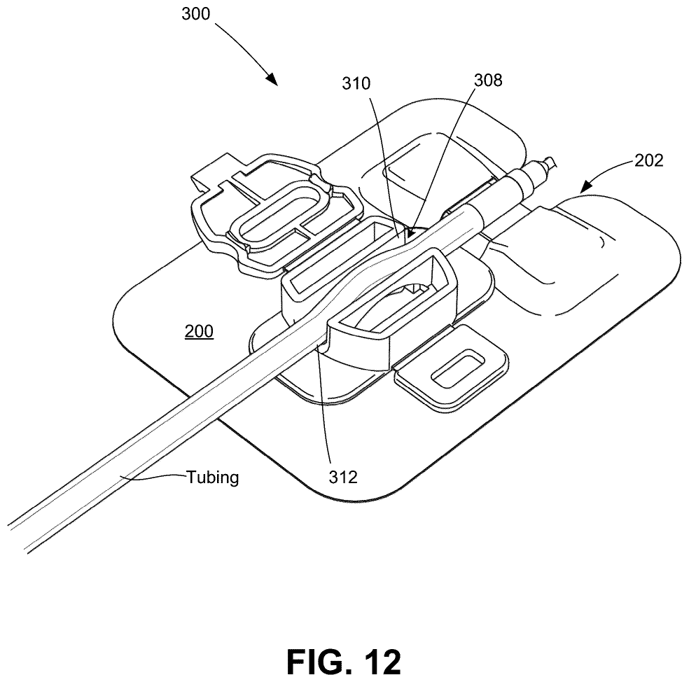

FIG. 12 shows the device 300 of FIGS. 9-11 placed on the patient near the infusion site with a portion of tubing (that runs from the infusion port outside of the patient's body) within a trench 308 of the device 300. With the support structure 200 of the device 300 stuck to the patient's skin near the infusion site, the infusion site is exposed to the eyes of a medical professional or other person, and the butterfly wings of the infusion port are secured down to the patient's skin by part at least a portion of the support structure 200. The opening 202 in the support structure 200 receives and secures the infusion port, as seen in FIG. 12. It is noted that the medical professional or other person that places the tubing within the trench 308 does not need to push down into or onto the patient with much, or possibly any, force at all to place the tubing within the trench 308, and this is because of, and a benefit that results from, the device 300 having the movable member 314, with the end result being much more comfort for the patient as opposed to known tubing retention devices that are available commercially and that typically require a greater amount of force to be used to get the tubing into a trench of the device.

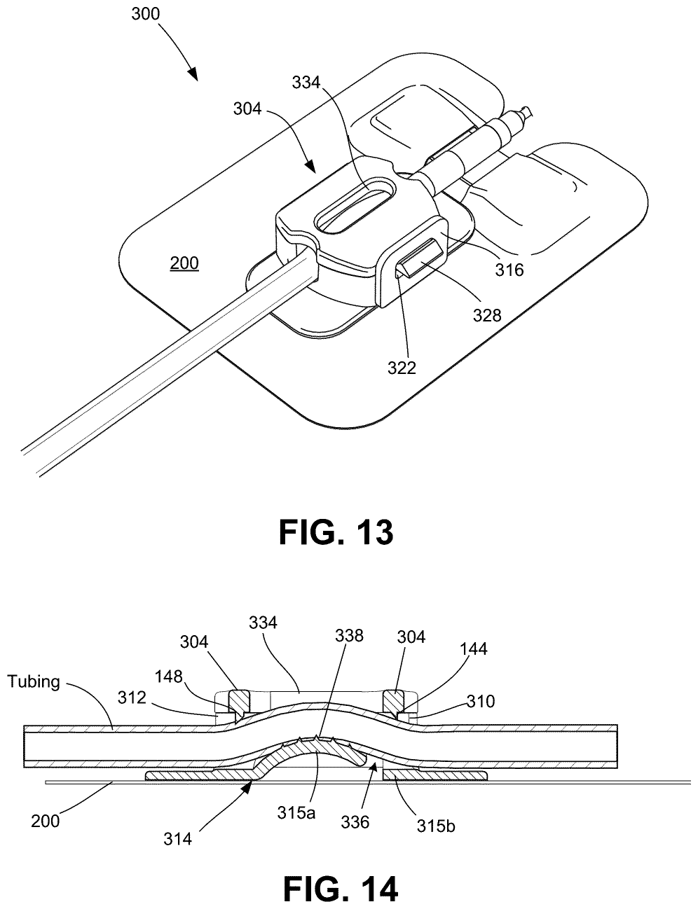

FIG. 13 shows the device 300 of FIGS. 9-12 with its cover 304 closed to retain that portion of the tubing within the trench 308 of the device 300. With the cover 304 of the device 300 closed, as shown in FIG. 13, the viewing window 334 shows the tubing retained within the trench 308. Once the cover 304 is placed over the base 302, as shown in FIG. 13, the person closing the cover 304 of the device 300 will hold the cover 304 down on the base 302 with one hand and, either with that same hand or with their other hand, will move the hinged securement tab 316 so that the clip 328 of the cover 304 passes through the aperture 322 formed in the body 318 of the hinged securement tab 316. With the cover 304 closed in this way, the cover 304 is held securely in its closed position, and it is unlikely that any force pushing up on the cover 304 by the tubing retained in the trench 308 will result in the cover 304 unexpectedly or unintentionally popping open. Also, the hinged side movement of the tab 316 to get the clip 328 to enter and be retained within the aperture 322 in the tab 316 means that force to close the cover 304 is a side force by the person placing the device 300, and this closing side force is easier for the placing person to apply as well as more comfortable for the patient on which the device 300 has been placed. With known retainer devices, a downward force (into the patient and the patient's skin) is required which is harder for the placing person to do and is less comfortable to the patient.

FIG. 14 is similar to FIG. 8 but FIG. 14 shows the device 300 of FIG. 13 in cross section and not the device 100 in cross section. The device 300 shown in FIG. 14 has the type of movable member 314 seen in FIGS. 9-13. The movable member 314 of FIG. 14 can be seen and understood to be a bridge with one long piece 315a separated by a slit or separation 336 from the other end of the bridge 315b. This can be viewed as a bridge with one connection point on one end of the bridge, as is shown in FIGS. 8 and 14, versus a bridge with two connection points on each end of the bridge as is shown in FIG. 7.

In FIG. 14, a force pushing down on the one long piece 315a of the bridge will cause that long piece 315a to move down (toward the support structure 200) further into the trench 308, at least until the space of the slit 336 is closed by the downward movement of that one long piece 315a. This movability of the movable member 314 (like the movability of the one-piece bridge 126b of FIG. 8 and the two-piece bridge 126a of FIG. 7) is important to the device 300 being able to accommodate any one of a variety of different-diameter tubes (whether catheters or other types of medical tubing). A device 300 with the movable member 314 can accommodate and hold secure tubing with a certain maximum outer diameter, with the cover 304 closed and not popping open, and that same device 300 with that movable member 314 also can accommodate and hold secure tubing with an outer diameter less than that maximum diameter. There is a range of outer diameters of tubing that such a device 300 can accommodate and hold secure, and this then means that a different specific device 300 does not have to be made for each specific tubing outer diameter. For example, one exemplary device according to the invention could hold and secure the tube of a Foley catheter with an outer diameter of 5 millimeters, and that very same device also could hold and secure the tube of a Foley catheter with an outer diameter of 6 millimeters as well as any other tube of a Foley catheter with an outer diameter between 5.0 mm and 6.0 mm. Other tubing outer diameter ranges can be accommodated by an exemplary inventive device such as, for example, 3 millimeters to 5 millimeters, or, as another example, 5 millimeters to 7 millimeters, or, as still other examples, any of the tube outer diameter ranges given above (whether expressed in millimeters or French units).

The one long piece 315a of FIG. 14 can have one or more gripping members 338 on it, and each gripping member 338 can be oriented in a direction that is left, up, or right, depending on the type or direction of gripping that is desired for each of the gripping members 338. The gripping members 338 shown in FIG. 14 are pointed generally in the up direction.

While not shown in FIGS. 9-14 in connection with the device 300 and its associated support structure 200, a two-piece bridge, like the two-piece bridge 126a of FIG. 7, could be used in the trench 308 of the device 300 instead of the one-piece bridge 314 shown in FIG. 14. With respect to a two-piece bridge, whether used in connection with the device 100 of FIG. 1 or the device 300 of FIG. 9, it is noted that the two-piece bridge does not have to have the slit or separation located directly in the middle of the two bridge pieces. That is, in a two-piece bridge arrangement of the movable member, the slit or separation or space between the two bridge pieces could be located anywhere on the length of the arched bridge where that length goes from one of the arched bridge end points to the other/opposing arched bridge end point.

Regardless of the type of movable member that is used in the device 300 of FIG. 9, it is noted that the cover 304 has the two channel brackets 330, 332 that will be aligned with the first and second ends 310, 312 of the trench 308 when the cover 304 is closed. The purpose of the first and second channel brackets 330, 332 can be seen with reference to FIG. 14. The channel brackets 330, 332 contact the tubing and push down on or into a portion of the wall of the tubing to hold it in place in the trench 308 when the cover 304 of the device 300 is closed. That force pushing down on the tubing by the closed cover 304 and the channel brackets 330, 332 also causes the tubing to push down on the movable member 314 of the device 300. In addition to the channel brackets 330, 332 contacting and pushing down on or into the wall of the tubing when the cover 304 is closed, the channel brackets 330, 332 help to prevent longitudinal movement of the tubing because of how they contact and/or dig into a portion of the wall of the tubing, as shown in FIG. 14.

With reference still to FIG. 14, it is noted that the movable member 314 can have one or more of the protrusions, barbs, or gripping members 338 on its surface that face/contact the tubing. Such gripping members 338 can perform a similar function as the channel brackets 330, 332, in that the gripping member(s) 338 can contact and push up on or into a portion of the wall of the tubing to hold the tubing in place within the trench 308 and to prevent longitudinal movement of the tubing within the trench 308. As stated above, each gripping member 338 can be oriented in a direction that is left, up, or right, depending on the type or direction of gripping that is desired for each of the gripping members 338. The gripping members 338 shown in FIG. 14 are pointed generally in the up direction.

The embodiment shown and described herein with reference to FIGS. 1-8 shares many features and aspects of the embodiment shown and described herein with reference to FIGS. 9-14, and it should be understood that one or more features and aspects of one embodiment can be applied to the other embodiment even if not expressly stated as such herein. Also, still other embodiments of devices and methods according to the invention are to be considered included herein even if not expressly stated or shown given that the invention is not limited only to the specific details or embodiments disclosed herein.

1. A single-piece device for securing a portion of tubing, the tubing for use in connection with medical treatment of a patient, the device comprising:
a section configured to receive the portion of the tubing;
a member configured to move when at least a portion of the portion of the tubing received by the section is pressed against the member; and
a cover configured to apply pressure to keep the portion of the tubing within the section when the cover is closed.

2. The single-piece device of claim 1 wherein the tubing is a catheter.

3. The single-piece device of claim 1 wherein the device is formed by injection molding.

4. The single-piece device of claim 1 wherein the member moves by being deflected from a first position to a second position as a result of the pressure applied to the portion of the tubing within the section when the cover is closed.

5. The single-piece device of claim 1 further comprising a male clip and a female tab that are configured to be connected mechanically to hold the cover closed.

6. A catheter stabilization device comprising a movable member configured to accommodate separately a first catheter of a first outer diameter and a second catheter of a second outer diameter that is different from the first outer diameter.

7. The catheter stabilization device of claim 6 wherein the movable member is formed integrally with and as part of the device by injection molding.

8. The catheter stabilization device of claim 6 further comprising a support structure configured to be attached directly to skin of the patient.

9. The catheter stabilization device of claim 8 wherein the support structure is ultrasonically welded to at least one part of the device.

10. A method of securing tubing that is used in connection with medical treatment of a patient, the method comprising:
attaching a tubing securement device to skin of the patient, the device comprising a support structure configured to be attached directly to the skin of the patient, a section configured to receive a portion of the tubing, a member configured to move when at least a portion of the portion of the tubing received by the section is pressed against the member, and a cover configured to apply pressure to keep the portion of the tubing within the section when the cover is closed;
placing the portion of the tubing in the section of the device; and
closing the cover of the device to keep the portion of the tubing within the section of the device.
